(19) United States
(12) Patent Application Publication
(10) Pub. No.: US 2010/0307230 A1
(43) Pub. Date: Dec. 9, 2010

(54) METHOD AND SYSTEM FOR MONITORING THE STATE OF AN ENGINE OIL IN AN INTERNAL COMBUSTION ENGINE

(75) Inventors: Markus Gilch, Mauern (DE); Olaf Graupner, Kofering (DE)

Correspondence Address: COHEN, PONTANI, LIEBERMAN & PAVANE LLP, 551 FIFTH AVENUE, SUITE 1210, NEW YORK, NY, 10176, US

(73) Assignee: Continental Automotive GmbH, Hannover (DE)

(21) Appl. No.: 12/310,422

(22) PCT Filed: Aug. 20, 2007
(86) PCT No.: PCT/EP2007/058624

§ 371 Date: May 12, 2009

(30) Foreign Application Priority Data
Aug. 21, 2006 (DE) 102006038968.9

(51) Int. Cl. G01N 33/28

(52) U.S. Cl. 73/114.55

(57) ABSTRACT

A method for monitoring the state or an engine oil in an internal combustion engine, in which first the viscosity of the engine oil is determined at different temperatures of the engine oil, after which a viscosity index is ascertained from the viscosity and the temperature of the engine oil, which method permits a reliable statement about the state of the engine oil during its issue in the internal combustion engine to be made, the viscosity index of the engine oil is determined at different successive times and the change of the viscosity index over time is used as a measure for assessing the oil state.

The invention relates to a method for monitoring the state of an engine oil in an internal combustion engine as claimed in the preamble of patent claim 1.

The need to monitor the filling level of the engine oil is known from automobile technology. Furthermore, it is known that the quality of the oil deteriorates with time, particularly owing to contamination. For this reason, the engine oil is usually replaced during regular maintenance work on the motor vehicle. Since the state of the engine oil depends, inter alia, also on the driving style of the motor vehicle user, there is an increasing trend to monitoring the oil state separately and instruct the user that an oil change has to take place.

To date, as accurate as possible a prediction of the time for an oil change has essentially been performed by measuring the conductivity or the permittivity of the oil using capacitive methods. Known designs using the capacitive method can simultaneously be used to measure filling level given that the electrode structure is designed as concentric tubes. However, these have the disadvantage that water drops or metal particles contained in the oil can short circuit the narrowly spaced-apart electrode structures, thus causing permanent malfunctions. Experience in this field has also shown that unambiguous statements on the service life and the state of an oil are impossible with purely capacitive sensors.

It is known from DE 103 45 253 A1 to introduce a piezo-oscillator into an oil circuit and use it to determine the viscosity of the oil. The viscosity alone is, however, inadequate as a basis for determining the oil state, since different aging phenomena can certainly exert opposite influences on the viscosity of the engine oil. For example, a soot intrusion thickens the oil, whereas a fuel intrusion thins the oil. If there is now simultaneously an intrusion of fuel and of soot, the viscosity of the oil can remain unchanged in some circumstances but in this case the state of the oil deteriorates substantially owing to the soot and the fuel. In the case of a pure viscosity measurement, the deteriorated state of the oil would remain undetected, which would lead to severe wear in the internal combustion engine. Consequently, an observation of the viscosity of the engine oil does not lead to a reliable judgment of the state of the engine oil. Again, a viscosity measurement does not enable a reliable statement on the quality of a newly filled engine oil. Filling the internal combustion engine with a low-grade engine oil is not detected by a viscosity measurement.

It is now the object of the invention to specify a method for monitoring the state of an engine oil that permits a reliable statement on the state of the engine oil during its use in the internal combustion engine.

This object is achieved according to the invention by a method having the features specified in patent claim 1.

An informative judgment of the quality of the engine oil during operation of the vehicle is possible by determining the viscosity index at different successive instants (t0, t1, t2) and by assessing the oil state over time through a measurement of the variation in the viscosity index. During each warm-up period of the internal combustion engine, the viscosity behavior of the engine oil can be acquired in a large temperature interval (for example between 15° C. and 100° C.), and the current viscosity index of the engine oil can be determined therefrom. The viscosity index thus determined varies after each warm-up period of the engine, for example through the intrusion of soot or fuel into the engine oil, or through the decomposition of viscosity index improvers. The simultaneous intrusion of soot and fuel into the engine oil can lead to the viscosity remaining unchanged, although the viscosity index is varying measurably. Also, the destruction of the viscosity index improvers that, as long-chain molecules, are subject to a rapid decomposition, is reliably detected by the inventive method, something which would not be possible with a pure viscosity measurement. A high quality statement on the instantaneous quality of the engine oil in the internal combustion engine is rendered possible by the measuring of the variation in the viscosity index.

In one refinement, the viscosity index of the new engine oil is firstly determined, and then the deviation from this viscosity index is assessed. A new engine oil is filled during an oil change, and this can be detected electronically by an oil level sensor. The new engine oil prescribes a measured value for the viscosity index, which represents a good judgment scale for the engine oil quality. It is also conceivable for the vehicle manufacturer to permit various engine oils with different viscosity indices. The first measurement of the viscosity index now yields a reliable reference value for judging the aging of the engine oil when changing from one engine oil to the other. Moreover, by stipulating a minimum viscosity index it is possible to reliably detect whether a low-grade engine oil has been filled into the engine when the filled engine oil undershoots this value. This information is of great importance in the judgment of warranty claims, in particular.

In one development, the ascertained viscosity indices are stored in an electronic memory. A simple electronic processing of the measurement results is thereby enabled. Moreover, the information relating to the viscosity indices that the engine oil exhibited at specific instants is retained, and this can, for example, be an important item of information for judging warranty cases in the event of engine damages.

When an instruction is output to a vehicle driver upon a prescribed deterioration in the state of the engine oil, said vehicle driver can occasion an oil change if necessary. If the vehicle driver has not followed this instruction quickly enough, reasons for excluding warranty services can result for the vehicle manufacturer. Economic and ecological advantages accrue with the complete usage of the engine oil up to its limit of wear.

In one development, the instruction to the vehicle driver is visible in the combined instrument. The vehicle driver is particularly alert to the combined instrument, and so the instruction relating to a poor oil state cannot be overlooked. Moreover, it is advantageous if the instruction is acoustically perceptible for the vehicle driver.

In one refinement, a recommendation for the residual running time of the engine oil is derived from the ascertained state of the engine oil. This is particularly important in order to be able to make optimum use of the engine oil from economical and ecological points of view. This residual running stretch, which can be shown in the vehicle display, represents a very important item of information for the driver, since he can plan a service in good time as the quality of the engine oil begins to be lost. In fact, an engine oil only begins to age extremely rapidly chemically once a certain aging threshold is reached. Within a very short remaining residual running stretch, this then leads to a complete breakdown of the oil, which is expressed by formation of black sludge and leads to engine damage. Until this critical aging state is reached, however, the oil can appear completely inconspicuous if thickening and thinning effects are superposed. If this critical limiting value is overshot, there is an immediate need for a stay in the workshop. If this occurs without advance warning, in the most unfavorable case the driver can be surprised by it while he is on a lengthy journey. Consequently, a reliable prediction of a residual running stretch is very advantageous.

In one development, the recommendation for the residual running time of the engine oil is displayed to the vehicle driver. If, for example, the vehicle driver is facing a long vacation trip, he can estimate very well whether he should undertake the oil change before or after the journey.

In a further refinement, the vehicle manufacturer prescribes a minimum viscosity index upon whose undershooting a low-grade engine oil is inferred. This is of great significance for the claims within the scope of a warranty. If it can be demonstrated that vehicle driver has used an impermissible engine oil of poor quality, it is conceivable to exclude a warranty service.

The invention is explained below with reference to the drawing, in which:

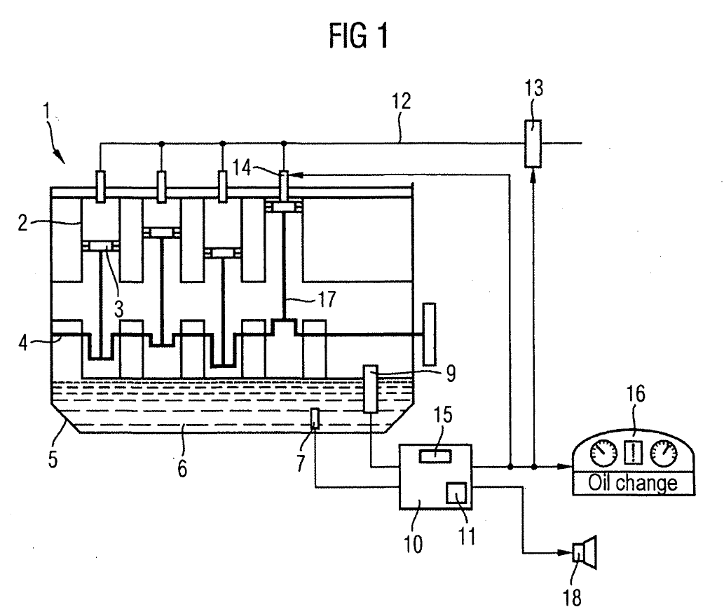

FIG. 1 shows an internal combustion engine with four cylinders,

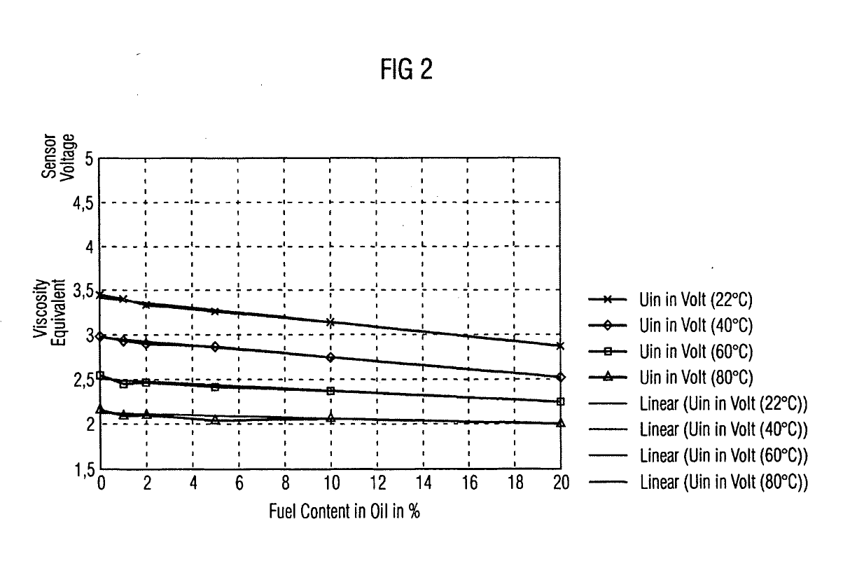

FIG. 2 shows the output signal of the viscosity sensor for various fuel concentrations in the engine oil,

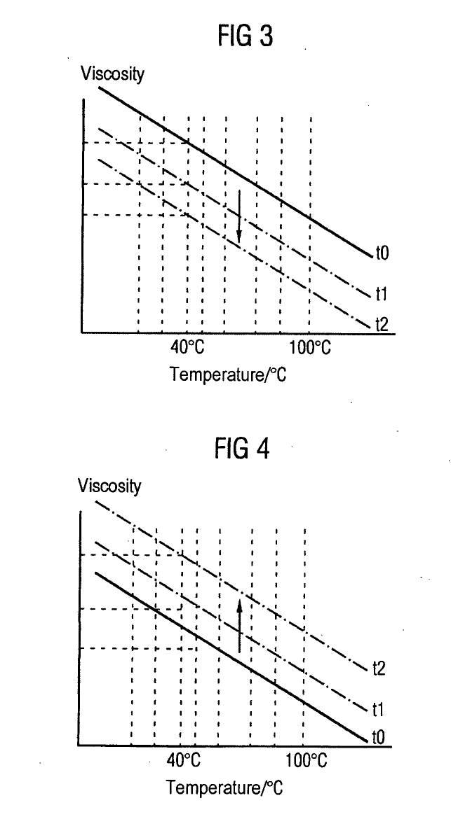

FIG. 3 shows the viscosity of an engine oil as a function of temperature,

FIG. 4 shows the behavior of the viscosity of an engine oil as a function of temperature and for the intrusion of soot,

FIG. 5 shows the viscosity behavior of an engine oil against temperature,

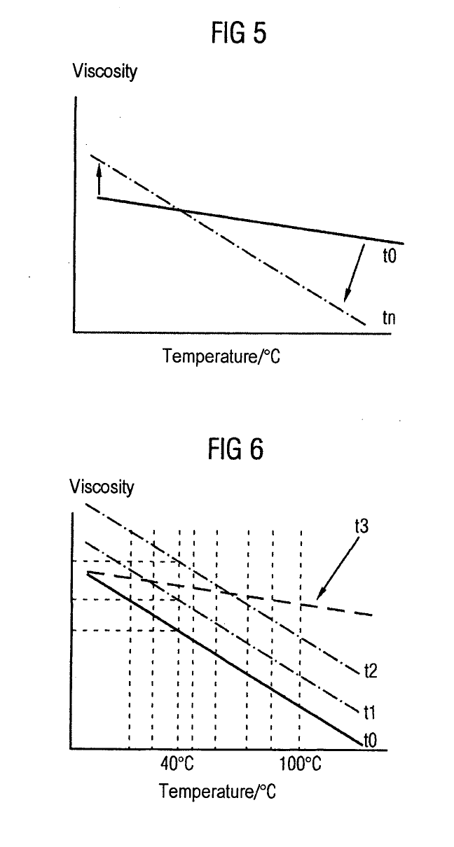

FIG. 6 shows, in turn, the viscosity of an engine oil against temperature,

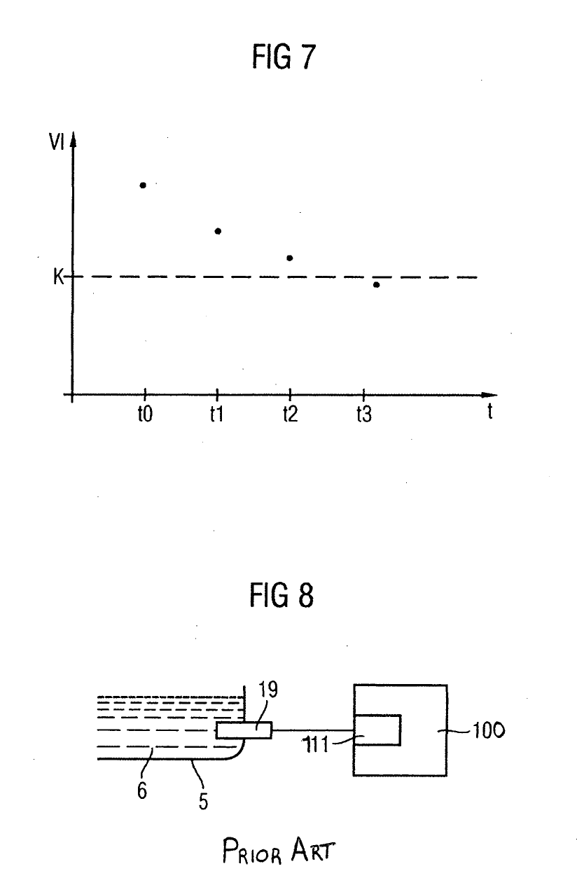

FIG. 7 shows the viscosity indices of an engine oil at various instants t₀ to t₃, and

FIG. 8 shows a method for determining the quality and the state of the engine oil in accordance with the prior art.

FIG. 1 shows an internal combustion engine 1 with four cylinders 2 in which pistons 3 are arranged. The pistons 3 are mounted movably in the cylinders 2 and connected to the crankshaft 4 by means of connecting rods 17. The movable parts illustrated here are lubricated by engine oil 6 during operation of the internal combustion engine 1. The engine oil 6 is located in the oil sump 5, and it is fed to the lubricating points via a pipeline system (not illustrated here).

The quality of the engine oil 6 deteriorates over the operating time of the engine oil 6 in the internal combustion engine 1. This deterioration takes place, for example, through the intrusion of fuels into the engine oil 6 or through the intrusion of soot into the engine oil 6 in the case of diesel engines. Such contaminants influence the viscosity of the engine oil 6, but in addition they also influence the ability of the engine oil 6 to protect the moving parts at the lubricating points. Additives present in the engine oil 6 can, for example, be decomposed, thus negatively influencing the quality of the engine oil 6. Such additives are, for example, the so-called viscosity index improvers. Viscosity index improvers (VI improvers) contain agents (viscosity improvers, polymers) that, when dissolved in the engine oil 6, improve the viscosity/temperature behavior, that is to say they reduce the temperature dependence of the viscosity. At low temperatures, they improve the flow behavior of the engine oil 6, and at high temperatures they effect a higher viscosity than without VI improvers. VI improvers are an important component in multigrade engine oils. The most important viscosity index improver groups are: polymethacrylates (PMAs), olefin copolymers (OCPs), polyisobuthylenes (PIBs) and styrene-butadiene copolymers (SCBs) or styrene-isoprene copolymers (SICs).

A temperature sensor 7 can be used to acquire the temperature of the engine oil 6, and a viscosity sensor 9 can be used to acquire the viscosity of the engine oil. The two values can be fed to a control device 10 in which a microcontroller 11 and an electronic memory 15 are present. From the measured values of the viscosity and the temperature of the engine oil 6, the microcontroller 11 can determine the viscosity index of the engine oil 6 at the measuring instant. This value can be stored in the electronic memory 15.

The viscosity index is preferably ascertained in the warm-up period of the internal combustion engine 1 by using the viscosity sensor 9 to determine the viscosity of the engine oil 6 at various temperatures of the engine oil 6. The viscosity index is formed as the quotient of viscosity and temperature of the engine oil 6. A high viscosity index means here that the viscosity of the engine oil 6 changes only very slightly as the temperature varies. A low viscosity index means, by contrast, that the viscosity of the engine oil 6 changes to a large extent as the temperature changes. A high viscosity index is advantageous at any rate, since with such an engine oil 6 the viscosity behaves approximately the same at all operating temperatures of the internal combustion engine 1. If the viscosity index drops steeply, this is an indication that the quality of the engine oil 6 has declined substantially. This can be ascertained by the control device 10, and appropriate countermeasures can be introduced. It is, for example, conceivable for the control device 10 to output to the injection pump 13 signals whose effect is that the speed of the internal combustion engine 1 no longer overshoots a certain maximum value. To this end, the injection pump 13 conveys a smaller fuel quantity to the injection valves 14 via the fuel line 12, the power of the internal combustion engine 1 thereby declining. In combination, or as an alternative thereto, a warning can be output in the combined instrument 16 indicating to the vehicle driver that an oil change is necessary. Moreover, it is also possible to conceive an acoustic warning to the vehicle driver via a loudspeaker 18 that instructs the vehicle driver to change the engine oil 6. Given the viscosity indices, measured values are available here that permit an accurate statement on the quality of the engine oil 6 during operation of the internal combustion engine 1. A statement on the viscosity such as is available owing to the viscosity sensor 9, is not adequate for a complete description of the quality of the engine oil 6, since, for example, the soot intrusion and fuel intrusion in combination can lead to an unchanging viscosity, although the quality of the engine oil 6 is substantially reduced. The deterioration in the engine oil state is reliably detected with the inventive method.

FIG. 2 shows the output signal of the viscosity sensor for various fuel concentrations in the engine oil 6. Measurement results are illustrated for various temperatures between 22° C. and 80° C.

FIG. 3 shows the viscosity of an engine oil 6 as a function of temperature. An engine oil 6 is illustrated that has been filled as new at instant t₀ into the internal combustion engine 1. Up to the instant t₁, the engine oil 6 is thinned by the intrusion of fuel, as a result of which the viscosity of the engine oil 6 has diminished, and this is illustrated by the dashed curve at t₁. A further intrusion of fuel up to the instant t₂ has the effect of again diminishing the viscosity of the engine oil 6, and this is illustrated by the curve at t2.

By contrast, FIG. 4 shows the behavior of the viscosity of an engine oil 6 as a function of the temperature upon intrusion by soot. The oil 6 newly introduced into the internal combustion engine 1 shows a behavior corresponding to the curve at t₀. The engine oil 6 has been thickened by the soot intrusion up to the instant t₁, and this leads to the curve at t₂. A further soot intrusion leads to further thickening of the engine oil 6, and this is illustrated by the curve at t₂. The effects from FIG. 3 and FIG. 4 can, however, be superposed on one another when both soot and fuel intrude into the engine oil 6, the result of which is that the viscosity does not vary, but the quality of the engine oil 6 is subjected to substantial deterioration. This is the reason that the pure measurement of the viscosity is inadequate for reliably determining the quality of the engine oil 6.

It is also possible to illustrate the deterioration in the oil quality through the decomposition of viscosity improvers (VI improvers). VI improvers are long-chain molecules that are added to the high grade engine oil in order to cause a flat profile of the viscosity index. With VI improvers, the viscosity of the cold engine oil changes only very slightly by comparison with that of the hot engine oil. It is precisely this behavior that is necessary for the optimum lubrication of the internal combustion engine in all operating states. An excessively viscous engine oil passes only very slowly to the lubricating points, whereas an excessively thin-bodied engine oil loses its lubricity. It is therefore optimal if an engine oil has the same viscosity at every operating temperature. This optimum is approached by the use of VI improvers. Unfortunately, the long-chain molecules of the VI improvers are destroyed during operation of the engine oil. This destruction of the VI improvers can be reliably detected with the inventive method. The VI improvers are an essential component of modern engine oils, and so it is possible to directly infer the state and the quality of the engine oil by detecting the destruction of the VI improvers.

To this end, the curve at t₀ in FIG. 5 shows the viscosity behavior of a new engine oil 6 against the change in the temperature. After destruction of viscosity index improvers, the viscosity of the engine oil 6 behaves in accordance with the curve illustrated at tₙ when plotted against the change in the temperature. It is to be seen that the viscosity of the engine oil 6 is very high after destruction of the VI improvers at low temperatures, whereas it is very low at high temperatures. This indicates a very poor quality of the engine oil 6, since the engine oil 6 is extremely viscous at the cold start, and is exceptionally thinned-bodied at the operating temperature of the internal combustion engine 1, and this can lead to inadequate lubrication of the bearings and movable parts in the internal combustion engine 1.

FIG. 6 shows, in turn, the viscosity against the change in the temperature, illustrated for various instants of the operation of an engine oil 6 in the internal combustion engine 1. At t₀, the new engine oil 6 was filled into the internal combustion engine 1, and the viscosity was raised at the instant t₁ and at the instant t₂ by intrusion of soot. The curve t₃ illustrates the viscosity behavior of another engine oil 6 that deviates clearly from the viscosity behavior of the engine oil previously illustrated. These measurements can be used by the vehicle manufacturer to detect, for example, whether the vehicle driver has used an engine oil 6 of lesser quality, from which it is possible to derive a loss of warranty claims.

FIG. 7 shows the viscosity index of an engine oil 6 that was determined at various instants t₀ to t₃. The viscosity index is a characteristic quantity of the viscosity/temperature behavior of oils. Its numerical value is greater the flatter the viscosity/temperature profile, and vice versa. A high viscosity index therefore characterizes a slight change in viscosity via the change in temperature. This means that an engine oil 6 with a high viscosity index is of higher quality than one with a low viscosity index. At the instant t₀, for example, a newly filled engine oil 6 has a relatively high viscosity index that drops in the course of the operating time of the engine oil 6 in the internal combustion engine 1, and undershoots a critical value K between the instants t₂ and t₃. At the instant t₃, at the latest, the vehicle driver should receive the instruction that the engine oil 6 is to be changed immediately. Adequate lubrication of moving parts of the internal combustion engine 1 is no longer ensured with a viscosity index below the critical value K. When a low oil quality has been detected, in order to protect the internal combustion engine 1 the control device 10 can ensure that a low fuel quantity is conveyed to the injection nozzles 14 via the injection pump 13 and the fuel lines 12, as a result of which the power of the internal combustion engine 1 is reduced. The power of the internal combustion engine 1 is reduced to the extent that this ensures an adequate lubrication of the moving parts until the vehicle driver attends to a change of the engine oil 6.

FIG. 8 shows a method for determining the quality and the state of the engine oil 6 according to the prior art. Arranged in an oil sump 5 filled with engine oil 6 is a sensor 19 that undertakes a resistance measurement or a permittivity measurement in the engine oil 6. The measured values for the resistance or for the permittivity are fed to the control device 10 and evaluated by the microcontroller 11. The state of the engine oil 6 is inferred from these measured values. This method for determining the quality and state of the engine oil is very inaccurate and therefore only poorly suited to reliable statements on the quality and the state of the engine oil 6.

1.-10. (canceled)

11. A method for monitoring engine oil state in an internal combustion engine, the method comprising:
determining a viscosity of the engine oil at a plurality of different temperatures;
determining a viscosity index based at least in part on the from the viscosity and the temperature of the engine oil,
wherein the steps of determining a viscosity index of the engine oil is performed at different successive times, and the engine oil state is based on a variation in the viscosity index over time.

12. The method for monitoring the state of the engine oil, according to claim 11, wherein a desired value for the viscosity index is stored in an electronic memory.

13. The method for monitoring the state of the engine oil, according to claim 11, wherein the viscosity index is determined when new engine oil is put in the internal combustion engine, and a deviation from this viscosity index is assessed.

14. The method for monitoring the state of the engine oil, according to claim 13, wherein the ascertained viscosity indices are stored in an electronic memory.

15. The method for monitoring the state of an engine oil, according to claim 11, wherein an instruction is output to a vehicle driver upon a prescribed deterioration in the state of the engine oil.

16. The method for monitoring the state of the engine oil, according to claim 15, wherein the instruction for the vehicle driver is visible in a combined instrument.

17. The method for monitoring the state of the engine oil, according to claim 15, wherein the instruction for the vehicle driver is acoustically perceptible.

18. The method for monitoring the state of the engine oil, according to claim 11, further comprising determining a residual running time of the engine oil from the ascertained state of the engine oil.

19. The method for monitoring the state of the engine oil, according to claim 18, further comprising displaying a recommendation for the residual running time of the engine oil to the vehicle driver.

20. The method for monitoring the state of the engine oil, according to claim 11, wherein a determined viscosity index below a predetermined minimum viscosity index indicates a low-grade engine oil.

21. The method for monitoring the state of the engine oil, according to claim 20, wherein a vehicle manufacturer presets the minimum viscosity index.

22. The method for monitoring the state of the engine oil, according to claim 11, further comprising limiting an amount of fuel provided to the internal combustion engine when the engine oil state reaches a preset limit.

23. A system for monitoring engine oil state in an internal combustion engine, the system comprising:
a temperature sensor configured to measure a temperature of the engine oil;
a viscosity sensor configured to measure a viscosity of the engine oil;
a control device coupled to the temperature sensor and the viscosity sensor, the control device configured to determine a viscosity index at different successive times,
wherein the engine oil state is based on a variation in the viscosity index over time.

24. The system for monitoring engine oil state in an internal combustion engine, according to claim 23, further comprising:
an injection pump configured to be controlled by the control device; and
an injection nozzle in fluid connection with the injection pump,
wherein the control device is configured to limit an amount of fuel pumped by the injection pump when the engine oil state reaches a preset limit.
